(19) United States
(12) Patent Application Publication
(10) Pub. No.: US 2010/0191267 A1
(43) Pub. Date: Jul. 29, 2010

(54) ROTARY NEEDLE FOR NATURAL ORIFICE TRANSLUMENAL ENDOSCOPIC SURGERY

(75) Inventor: William D. Fox, New Richmond, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/359,824

(22) Filed: Jan. 26, 2009

(51) Int. Cl. A61B 17/34

(52) U.S. Cl. 606/185; 604/272

(57) ABSTRACT

A translumenal access device including a cannula defining a first lumen. The cannula may be sized for insertion into a working channel of a flexible endoscope. The device includes a rotatable rotary needle positioned within the cannula. The rotary needle may define a lumen that houses a stylet. The distal end of the rotary needle may define a distal circular opening with a cutting edge. The rotary needle may be used to slice through tissue during a surgical procedure.

### BACKGROUND

Access to the abdominal cavity may be required for diagnostic and therapeutic endeavors for a variety of medical and surgical procedures. Historically, abdominal access has required a formal laparotomy to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including, without limitation, laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. The trocar must pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient (e.g., mouth, anus, vagina) are known as Natural Orifice Translumenal Endoscopic Surgery (NOTES)™ procedures. Medical instruments, such as graspers, may be introduced through the working channel of a flexible endoscope, which typically has a diameter in the range of about 2.5 to about 4 millimeters.

These minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

An issue typically associated with current techniques for accessing various body cavities is the risk that nearby organs may be accidentally injured by a cutting tool or penetrating device, such as an endoscopic needle. The physician normally cannot see anatomical structures on the distal side of the tissue layers when the endoscopic needle is being pushed through the tissue layers. The tissue also may “tent” when the needle is being pushed through the tissue. Once the puncture is made, the needle may rapidly advance through the tissue due to the build-up of energy. Therefore, there is a risk that adjacent organs may be accidentally injured by the penetrating device.

The foregoing discussion is intended only to illustrate some of the shortcomings present in the art at the time, and should not be taken as a disavowal of claim scope.

### FIGURES

The novel features of the various embodiments are set forth with particularity in the appended claims. The various embodiments, however, both as to organization and methods of operation, may be best understood by reference to the following description, taken in conjunction with the accompanying drawings as follows.

FIG. 1 illustrates a flexible endoscopic portion of one embodiment of an endoscope inserted into the upper gastrointestinal tract of a patient.

FIG. 2 is partial perspective view of a portion of the endoscope shown in FIG. 1.

FIG. 3 is a perspective view of one embodiment of a rotary needle.

FIG. 4 is a cross-sectional view of one embodiment of an endoscopic assembly.

FIG. 5 is a perspective view of one embodiment of a rotary needle.

FIG. 6 is a perspective view of one embodiment of a rotary needle.

FIG. 7 is a perspective view of one embodiment of a surgical instrument that is adapted for use with the embodiment of the endoscopic needle assembly of FIG. 4

FIG. 8 is a perspective cross-sectional view of the handle assembly of the endoscopic assembly of FIG. 7.

FIGS. 9A-9D are is progression of partial cross section side views of the endoscopic assembly of FIG. 3 penetrating the portion of tissue.

FIGS. 10A-10D are is progression of partial cross section side views of the endoscopic assembly of FIG. 3 penetrating the portion of tissue.

FIG. 11 is a handle assembly in accordance with one embodiment

FIG. 12 is a partial cross-sectional view of the handle assembly of FIG. 11.

### DESCRIPTION

Before explaining the various embodiments in detail, it should be noted that the embodiments are not limited in their application or use to the details of construction and arrangement of parts illustrated in the accompanying drawings and description. The illustrative embodiments may be implemented or incorporated in other embodiments, variations and modifications, and may be practiced or carried out in various ways. For example, the rotary needle and endoscopic assembly configurations disclosed below are illustrative only and not meant to limit the scope or application thereof. Furthermore, unless otherwise indicated, the terms and expressions employed herein have been chosen for the purpose of describing the illustrative embodiments for the convenience of the reader and not to limit the scope thereof.

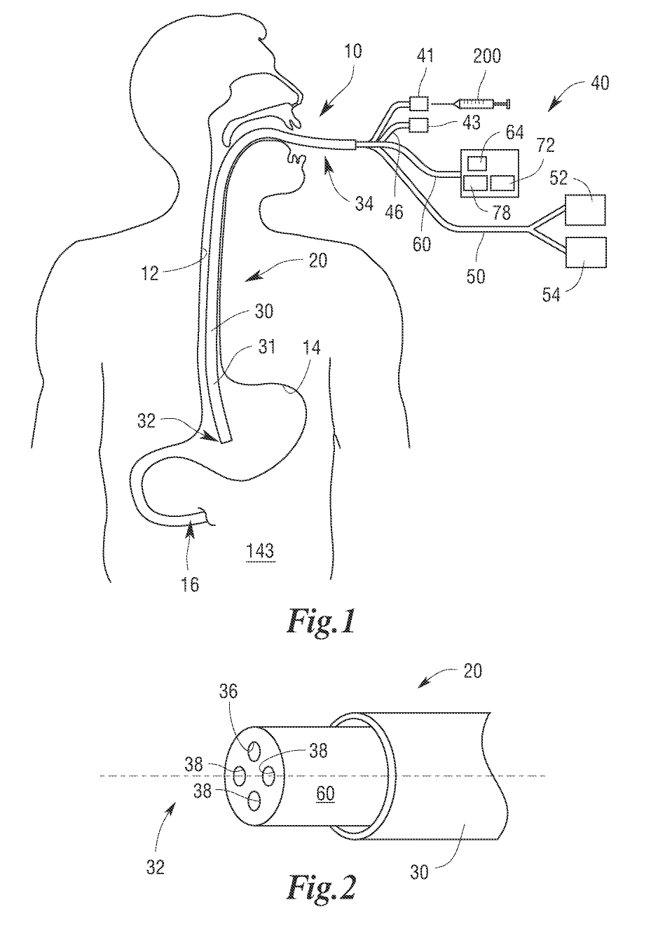

Newer procedures have developed which may be even less invasive than the laparoscopic procedures used in earlier surgical procedures. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as NOTES™. NOTES™ is a surgical technique whereby operations can be performed using any natural opening, such as trans-orally (as depicted in FIG. 1), trans-anally, and/or trans-vaginally.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined with the features of other embodiments. Such modifications and variations are intended to be included within the scope of the claims.

It will be appreciated that the terms “proximal” and “distal” are used herein with reference to a clinician gripping the surgical instrument. It will be further appreciated that, for convenience and clarity, spatial terms such as “top” and “bottom” also are used herein with respect to the clinician gripping the handle. However, surgical instruments are used in many orientations and positions, and these terms are not intended to be limiting and absolute.

FIG. 1 illustrates a flexible endoscopic portion 31 of an endoscope 60 (e.g., gastroscope) inserted into the upper gastrointestinal tract of a patient. FIG. 2 is a drawing of the distal portion 32 of the endoscope 60. FIG. 1 illustrates, in general form, one embodiment of a surgical instrument 20 that can be inserted through a natural orifice such as the mouth 10 and esophagus 12 into the stomach 14 to establish a surgical opening in the stomach 14 for performing a surgical operation such as a gall bladder removal, or a cholecystectomy, for example. As shown in FIG. 2, the surgical instrument 20 may comprise a hollow outer sleeve 30 that has a distal end 32 and a proximal end 40 (FIG. 1). In various embodiments, the hollow outer sleeve 30 may be fabricated from, for example, nylon or high-density polyethylene plastic. In various embodiments, the hollow outer sleeve 30 can serve to define various tool-receiving passages 38, or “working channels,” that extend from the natural orifice 10 to the surgical site. In addition, the hollow outer sleeve 30 may serve to define a viewing port 36. An endoscope 60 (FIG. 1) may be used for viewing a surgical site within the patient's body. Various cameras and/or lighting apparatuses may be inserted into the viewing port 36 of the endoscope 60 to provide the surgeon with a view of the surgical site.

As shown in FIG. 1, in various embodiments, one of the tools or surgical instruments that can be accommodated in the tool-receiving passage 38 is a hollow vacuum/air tube 50 that may communicate with at least one of a vacuum source 52 and a source of pressurized air 54. In one embodiment, the vacuum/air tube 50 can be sized to receive therein another surgical instrument in the form of the endoscope 60. A variety of different types of endoscopes are known and, therefore, their specific construction and operation will not be discussed in great detail herein. In various embodiments, the endoscope 60 may operably support a video camera that communicates with a video display unit 64 that can be viewed by the surgeon during the operation. In addition, the endoscope 60 may further have a fluid-supply lumen therethrough that is coupled to a source of water 72, saline solution, and/or any other suitable fluid and/or an air supply lumen that is coupled to the source of air 78.

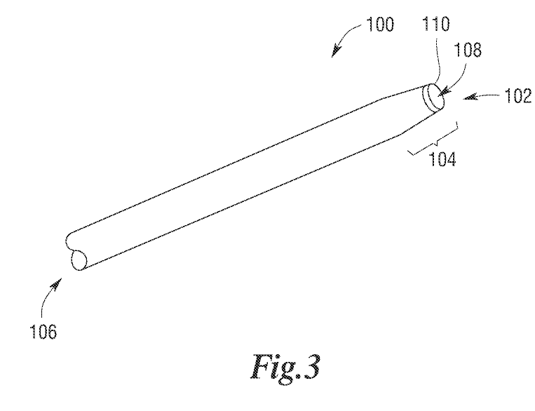

FIG. 3 illustrates one embodiment of a rotary needle 100. In various embodiments, the rotary needle 100 may be formed of a flexible tube which may have a channel or lumen extending from a proximal end 106 of the rotary needle 100 to a distal end 102 of the rotary needle 100. In one embodiment, the rotary needle 100 may be hollow. The distal end 102 of the rotary needle 100 may comprise a tapered portion 104. The tapered portion 104 may define an opening 108. In one embodiment, the opening 108 at the distal end 102 is circular. An edge 110 of the opening 108 may comprise a cutting surface, such that the periphery of the opening 108 has a sharpened cutting edge. The edge 110 may be formed using any suitable technique, such as cutting or grinding, for example. The rotary needle 100 may be fabricated from medical grade stainless steel, nitinol, or polyetheretherketon (PEEK) hypodermic tubing or any other suitable medical grade material, which may include metal and/or plastic suitable for medical applications, for example. Alternatively, the rotary needle 100 may be formed from an alternate type of metallic or polymeric tube and attached to a tube (not shown) such as by bolting, screwing, welding, crimping, gluing or any other suitable method. The rotary needle 100 may have an outer diameter in the range of about 0.010 inches to about 0.050 inches. For example, the rotary needle 100 may be formed from nitinol having an outer diameter of approximately 0.035 inches. The rotary needle 100 may have an inner diameter in the range of about 0.005 inches to about 0.045 inches. For example, the rotary needle 100 may have an inner diameter of 0.020 inches.

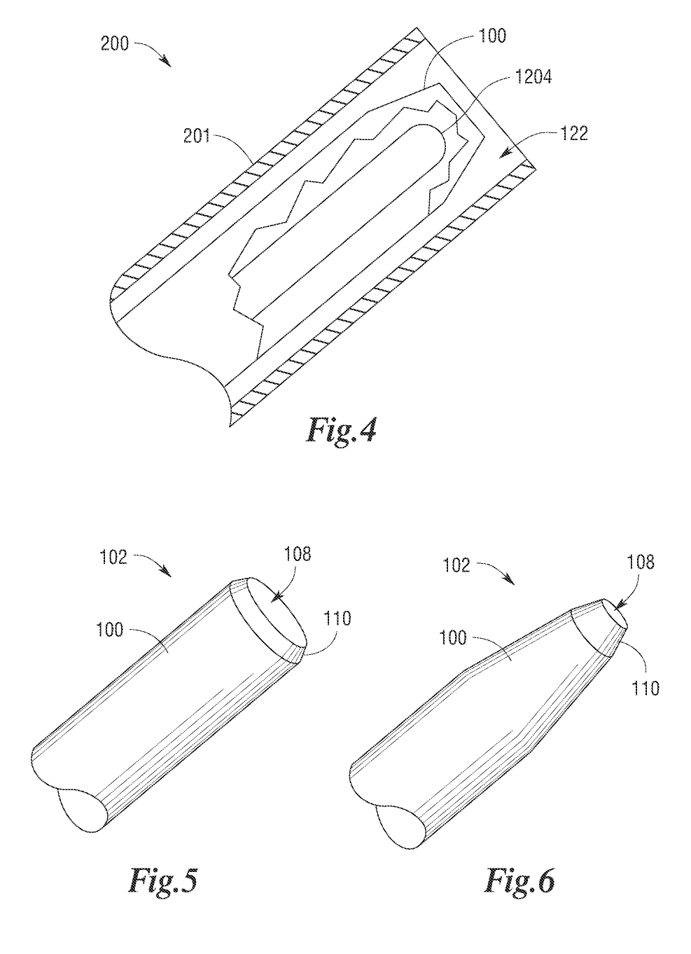

FIG. 4 is one embodiment of an endoscopic assembly 200 comprising the rotary needle 100, described in FIG. 3. In various embodiments, the endoscope 60 may comprise the one or more working channels 38 (FIG. 2) extending therethrough for receiving various instruments such as the endoscopic assembly 200, for example. The endoscopic assembly 200 may comprise a flexible cannula 201 (shown in cross-section). The cannula 201, or catheter, may comprise a central lumen 122 and a secondary lumen (not shown). The cannula 201 may be fabricated from nylon, polyvinylchloride (PVC), urethane, or any other suitable polymer. The endoscopic assembly 200 may further comprise, for example, the rotary needle 100 (shown with partial cutaway). The cannula 201 may be configured to retain the rotary needle 100. The rotary needle 100 may be slidably disposed within the central lumen 122 of the cannula 201. The secondary lumen of the cannula 201 may be in fluid communication with an inflatable member (not shown). The secondary lumen may be configured to provide fluid to the inflatable member located on, or near, the cannula 201.

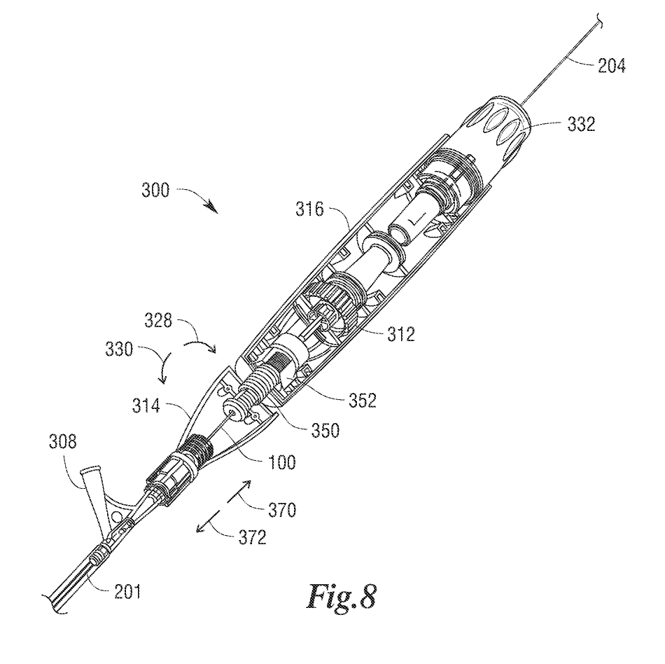

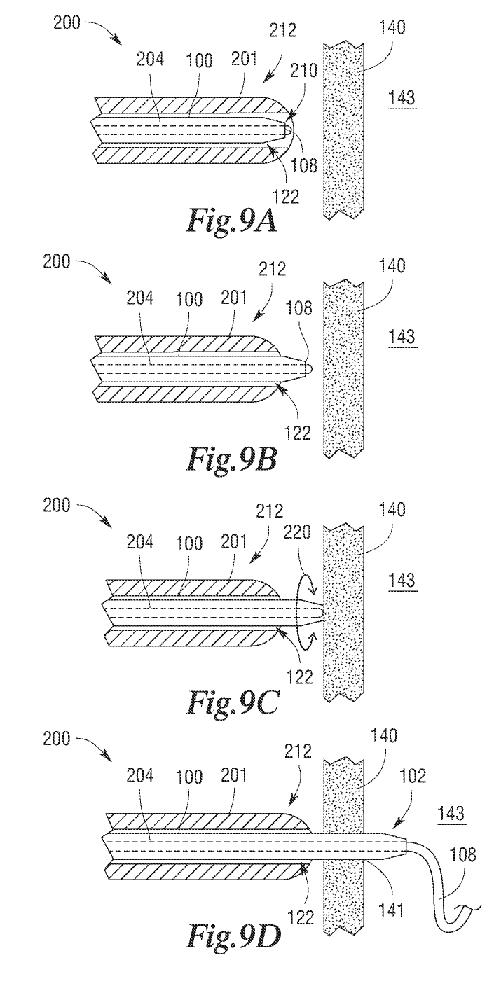

In various embodiments, the endoscopic assembly 200 may comprise a stylet 204. The stylet 204 may be fabricated from nytenol, or any other suitable material, with a TEFLON®, or any other suitable coating, placed upon the stylet 204. In various embodiments, the distal end of the stylet 204 may be formed with a blunt tip to prevent the stylet 204 from puncturing tissue 140 (FIGS. 9A-D). The stylet 204 may be flexible enough to travel along the length of the flexible endoscope 60 (FIG. 1). The operator may control the stylet 204 from the proximal end of the endoscope 60. As shown in FIG. 8, this proximal end of the stylet 204 may extend from a grip portion 316. The operator may have the ability to extend the stylet 204, or to move the stylet 204, distally. In addition, the operator may have the ability to retract the stylet 204, or move the stylet 204, proximally. The inflatable member may comprise an expandable balloon, pouch, or bag that extends around, and may be attached to cannula 201 with an adhesive such as cyanoacrylate, epoxy resin, or light-activated glue, or any other suitable attachment means. The rotary needle 100 may be formed from a flexible tube defining a central channel, or lumen. The central channel of the rotary needle 100 may be configured to allow the stylet 204 to extend from the proximal end of the rotary needle 100 through the distal end of the rotary needle 100. In one embodiment, the user may selectively extend and retract a distal portion of the stylet from the opening 108 of the rotary needle 100.

It is appreciated that the distal end 102 of the rotary needle 100 may be configured in a variety of embodiments. For example, as illustrated in FIG. 5, the distal end 102 of the rotary needle may comprise a substantially flat cylindrical portion (e.g., untapered) as shown in FIG. 3. In one embodiment, the rotary needle 100 is substantially the same diameter its entire length and has an edge 110 at the opening 108 to aid in cutting. In various embodiments, the wall thickness of the rotary needle 100 is thin enough such that an edge 110 is not needed to aid in cutting. FIG. 6 illustrates an embodiment wherein the rotary needle 100 has a relatively long tapered distal end 102 and cutting edge 110 at the opening 108. Accordingly, the rotary needle 100 is not limited to any particular configuration of the distal end and/or cutting edge. It is appreciated that a variety of configurations may be used.

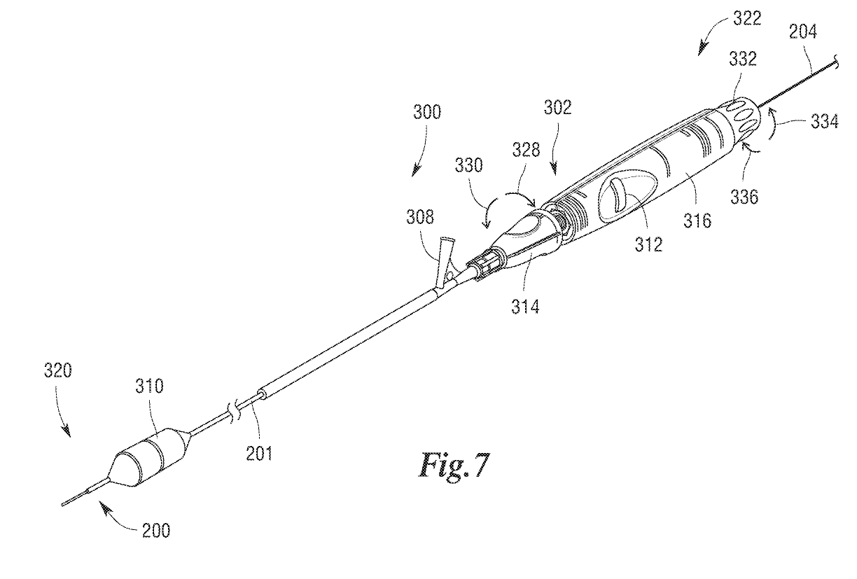

FIG. 7 is a perspective view of an embodiment of a surgical instrument 300 that is adapted for use with the endoscopic assembly 200. The surgical instrument 300 may include the cannula 201 attached to a handle assembly 302. The surgical instrument 300 may have a distal end 320 and a proximal end 322. The cannula 201 may be flexible and may be sized for insertion into the working channel of the flexible endoscope 60 (FIG. 1). The surgical instrument 300 may be used in conjunction with any suitable endoscopic assembly, such as those previously discussed. The rotary needle 100 may be located at the distal end 320 of the surgical instrument 300. The surgical instrument 300 is described next as it may be adapted for use with the endoscopic assembly 200, although the surgical instrument 300 may be adapted for use with various suitable endoscopic assemblies and tools.

The stylet 204 may extend through the cannula 201 and the handle assembly 302, such that a portion of the stylet 204 extends from the proximal end 322 of the handle assembly 302. The handle assembly 302 may include a luer connection 308 for delivering fluids to an inflatable member 310. As may be appreciated by those skilled in the art, the inflatable member 310 may be used to expand the opening created by the rotary needle 100 to allow for passage of the endoscope 60 through the tissue. The handle assembly 302 may include a wheel 312. A physician may operate the wheel 312 to rotate the rotary needle 100 to aid in penetrating the tissue 143 (FIGS. 9A-D). As may be appreciated by those skilled in the art, any suitable technique may be used to impart rotational movement onto the rotary needle 100 during operation. The handle assembly 302 also may include a distal portion 314. The distal portion 314 may be rotatable with respect to a grip portion 316. Rotation of the distal portion 314 in a first direction 328 may advance the rotary needle 100 from the cannula 201. Rotation of the distal portion 314 in a second direction 330 may retract the rotary needle 100 into the cannula 201. The handle assembly 302 may include a knob 332. Rotation of the knob 332 in a first direction 334 may engage the stylet 204 and prohibit distal or proximal movement of the stylet 204. Rotation of the knob 332 in a second direction 336 may disengage the stylet 204 and allow for a selected length of the stylet 204 to be manually advanced through the handle assembly 302, through the shaft 304, and into the body cavity 143 (FIGS. 1, 9A-D).

A partial sectional view of the handle assembly 302 is illustrated in FIG. 8. The rotary needle 100 may extend from the distal end 320 (FIG. 7) through the distal portion 314 and be axially secured to the grip portion 316. The rotary needle 100 may be coupled to the wheel 312, although those skilled in the art will appreciate that any suitable technique may be used to impart rotation. The distal portion 314 may be coupled to a threaded member 350, such as a nylon bolt. Rotation of the distal portion 314 causes the threaded member 350 to rotate. The threaded member 350 may be received by a nut 352 secured within the grip portion 316. Accordingly, rotation of the distal portion 314 by the user in the first direction 328 causes the threaded bolt 350 to feed into the nut 352 and move the distal portion 314 in a proximal direction 370. Rotation of the distal portion 314 by the user in the second direction 330 causes the threaded bolt 350 to feed out of the nut 352 and move the distal portion 314 in a distal direction 372.

The cannula 201 may be coupled to the distal end of the distal portion 314 by any suitable connection, such as gluing, welding, or a threaded connection, for example. Distal or proximal movement of the distal portion 314 also moves the cannula 201 in the distal direction 370 or the proximal direction 372, respectively. Since the proximal end of the rotary needle 100 may be coupled to the grip portion 316, the rotary needle 100 remains fixed in relation to the cannula 201. Moving the cannula in the distal direction 372 effectively retracts the distal end 102 (FIG. 3) of the rotary needle 100 into the cannula 201. The rotary needle 100 may be in this position, for example, when the endoscopic assembly 200 is being introduced to the access site. When the rotary needle 100 is retracted into the cannula 201, the likelihood of errantly cutting tissue with the rotary needle 100 is decreased. The user then may spin the distal portion 314 in the first direction 328 to move the cannula 201 in the proximal direction 370 and expose the distal end 102 of the rotary needle 100.

FIGS. 9A-9D are side views of one embodiment of the endoscopic assembly 200 as the assembly is used to access a body cavity 143, as also shown in FIG. 1. As illustrated in FIG. 9A, the stylet 204 (or “guide wire”) may be loaded inside a lumen of the rotary needle 100. The distal end 210 of the stylet 204 may extend from the opening 108 of the rotary needle 100. In one embodiment, the distal end 210 of the stylet 204 may initially extend approximately 0.04 inches beyond the opening 108. It will be appreciated that the user may adjust the position of the style 204 using the controls on the handle assembly 302. The stylet 204 may be biased, with a spring, for example, to partially extend from the opening 108. With the stylet 204 extending from the opening 108, the likelihood of cutting edge 110 from contacting tissue is decreased. If the bias is overcome, the stylet 204 may retract into the rotary needle 100.

FIG. 9B is a side view of the endoscopic assembly 200 with the rotary needle 100 extending from the distal end 212 of the cannula 201. In one embodiment, the user may selectively extend and retract a portion of the rotary needle 100 from the cannula 201 by rotation of the distal portion 314 of the handle assembly 302 (FIG. 7).

FIG. 9C is a side view of the endoscopic assembly 200 placed against a portion of tissue 140. The tissue 140 may be part of the stomach wall 14 (FIG. 1). As shown in FIG. 9C, the opening 108 of rotary needle 100 may be placed against the portion of the tissue 140 to be punctured by the rotary needle 100. As the rotary needle 100 is placed against the tissue 140, the bias on the stylet 204 is overcome, and the stylet 204 retracts into the rotary needle 100. It is appreciated that other techniques may be used to retract the stylet 204 into the rotary needle 100 prior to cutting. Once the rotary needle 100 is in proper position, the user may rotate the needle 100 to slice or cut through the tissue 140. The needle 100 may be rotated in a single direction, such as clockwise, or be rotated both clockwise and counter-clockwise, as indicated by arrows 220. The movement of the rotary needle 100 may be controlled by the operator of the surgical instrument. As the rotary needle 100 is rotated, the cutting edge 110 (FIG. 3) slices through the tissue 140.

FIG. 9D is a side view of the endoscopic assembly 200 with a portion of the rotary needle 100 penetrating the portion of tissue 140 through an opening 141. As the operator advances the rotary needle 100 distally, a portion of the rotary needle 100 may enter the body cavity 143, such as a peritoneal cavity. Once the tissue 140 has been penetrated, the stylet 204 may be advanced, such as by manual advancement, into the body cavity 143 by the user.

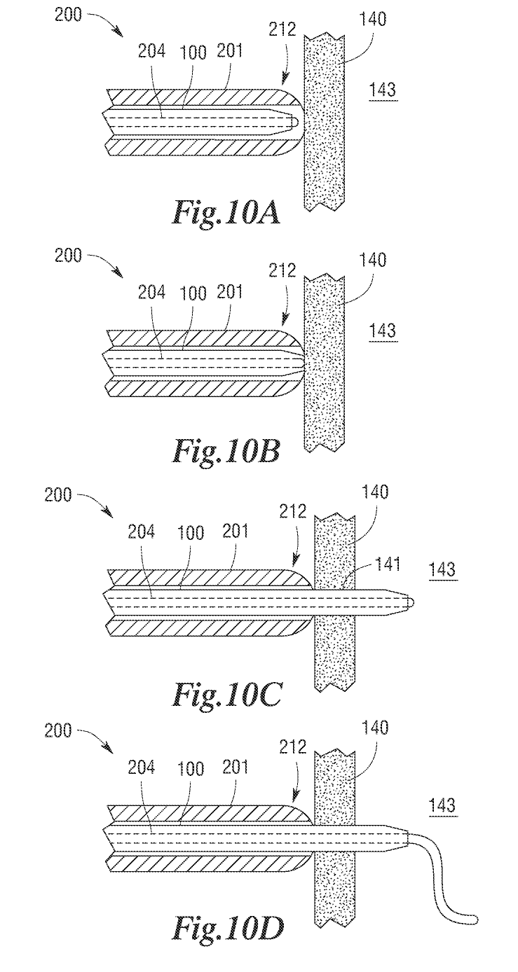

FIGS. 10A-10D are side views of one embodiment of the endoscopic assembly 200 as the assembly is used to access a body cavity 143, similar to FIGS. 9A-9D. As illustrated in FIG. 10A, the stylet 204 (or “guide wire”) may be loaded inside a lumen of the rotary needle 100. In one embodiment, as shown in FIG. 10A, the user may selectively place the distal end 212 of the cannula 201 against the tissue 140. As shown in FIG. 10B, the rotary needle may be advanced distally until it makes contact with the tissue 140. As illustrated, upon engagement with the tissue 140, the stylet 204 may retract into the rotary needle. For example, the engagement with the tissue 140 may overcome a spring bias of the stylet. It is appreciated that other techniques may be used to retract the stylet 204 into the rotary needle 100 prior to cutting.

Once the rotary needle 100 is in proper position, the user may rotate the needle 100 to slice or cut through the tissue 140. As may be appreciated, the seating the distal end 212 of the cannula 210 on the tissue 140 may assist in penetration of the tissue. For example, the cannula 210 may minimize shearing. FIG. 10C is a side view of the endoscopic assembly 200 with a portion of the rotary needle 100 penetrating the portion of tissue 140 through an opening 141. As the operator advances the rotary needle 100 distally, a portion of the rotary needle 100 may enter the body cavity 143, such as a peritoneal cavity. Once the tissue 140 has been penetrated, the stylet 204 may be advanced, such as by manual advancement, into the body cavity 143 by the user.

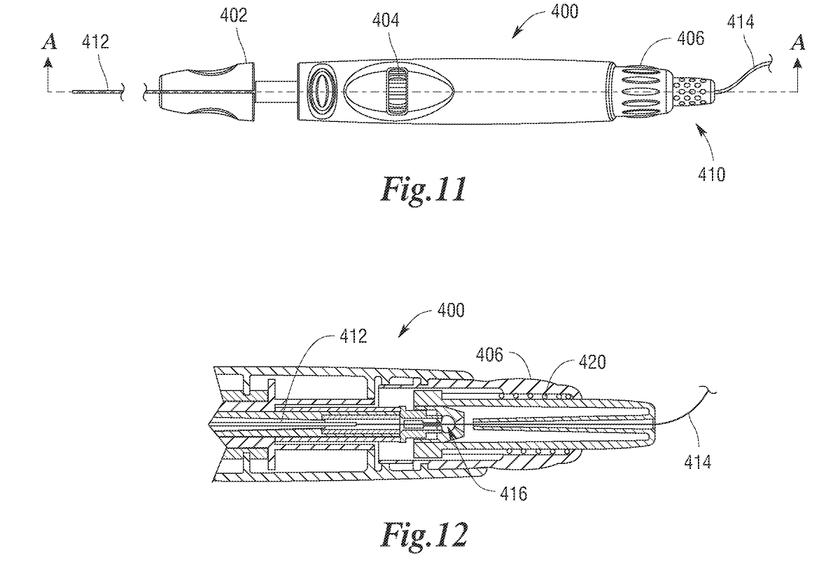

FIG. 11 illustrates a handle assembly 400 in accordance with one embodiment. The handle assembly 400 may be used in conjunction with any suitable endoscopic assemblies and tools, such as those previously discussed. As illustrated that handle assembly 400 may comprise a rotatable distal portion 402, a wheel 404, and a knob 406 at a proximal end 410. The handle assembly 400 may include the cannula 412 attached thereto. As previously discussed, a stylet 414 may extend through the handle assembly 400 and may be selectively advanced or retreated through the cannula 412 by an operator.

As illustrated in FIG. 12, which shows a partial cross section of FIG. 11 along A-A, the handle assembly may comprise a collett 416 that may selectively be coupled to the stylet 414. In various embodiments, rotation of knob 406 may engage the collett 416 to the stylet 414. When engaged, a biasing force from a spring 420, may be applied to the stylet 414. As may be appreciated, any suitable biasing member may be used, such as a coil spring, a leaf spring, a tension spring, a compression spring, for example. Furthermore, the biasing member may be located in any suitable location, such as in the handle assembly (as illustrated) or in the cannula, for example.

In operation, a user may selectively position the distal end of the stylet 414 with regard to the rotary cutter 100 (FIG. 4) by first loosening the knob 406, thereby loosening the collet 416. The stylet 414 may be distally or proximally moved with regard to the rotary cutter to a desired position. Once positioned, the collett 416 may be tightened through rotation of the knob 406. In one embodiment, the distal end of the stylet 414 may be positioned to extend approximately 0.04 inches beyond the opening 108 (FIG. 9A) or rotary cutter 100. Once the collet 416 is engaged, the spring 420 may provide a bias such that the relative position of the stylet 414 and the rotary cutter 100 are maintained. When the rotary cutter 100 and stylet 414 are pressed against an object, such as tissue, the spring bias may be overcome and the stylet may retract into the rotary cutter 100, thereby exposing the cutting edge.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments described herein will be processed before surgery. First, a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, or steam.

Although various embodiments have been described herein, many modifications and variations to those embodiments may be implemented. For example, different types of endoscopic assemblies may be employed. In addition, combinations of the described embodiments may be used. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A translumenal access device, comprising:
a flexible cannula defining a first lumen, the cannula sized for insertion into a working channel of a endoscope; and
a flexible rotatable rotary needle positioned within the cannula, the tubular needle defining a second lumen, the rotary needle comprising a distal end and a proximal end, the distal end of the rotary needle defining a distal opening comprising a cutting edge.

2. The device of claim 1, comprising a flexible stylet slidably disposed within the rotary needle, wherein the flexible stylet is selectably movable between a first position and second position.

3. The device of claim 2, wherein the flexible stylet is biased with a biasing member.

4. The device of claim 1, wherein the cutting edge is located on the periphery of the distal circular opening.

5. The device of claim 1, wherein the rotary needle position with respect to the cannula is selectable.

6. The device of claim 5, further comprising a handle assembly, wherein the rotary needle position is selected with controls on the handle assembly.

7. The device of claim 6, wherein the handle assembly comprises a wheel coupled to the rotary needle.

8. The device of claim 7, wherein the handle assembly comprises a movable distal portion.

9. The device of claim 8, wherein movement of the moveable distal portion moves the cannula with respect to the rotary needle.

10. A translumenal access device, comprising:
a handle assembly comprising a rotatable portion;
a cannula defining a first lumen, the cannula sized for insertion into a working channel of an endoscope, the cannula coupled to the rotatable portion;
a rotatable rotary needle positioned within the cannula, the tubular needle defining a second lumen, the rotary needle comprising a distal end and a proximal end, wherein the outer diameter of the rotary needle is less than about 0.050 inches; and
a stylet selectively positionable within the rotary needle, wherein the distal end of the rotary needle defines a distal circular opening.

11. The device of claim 10, wherein rotation of the rotatable portion in a first direction advances the distal end of the rotary needle from the cannula and rotation of the rotatable portion in a second direction retracts the distal end of the rotary needle into the cannula.

12. The device of claim 10, wherein the distal circular opening comprises a cutting edge.

13. The device of claim 10, comprising a wheel located in the handle assembly for rotating the rotary needle.

14. The device of claim 13, wherein the stylet is biased with a biasing member.

15. A method comprising:
inserting an endoscope into a lumen of a patient;
inserting a surgical instrument into the lumen of the patient through a working channel of the endoscope;
placing a cannula near a portion of tissue to be penetrated;
advancing a rotary needle distally from the cannula;
rotating the rotary needle and penetrating the tissue with a distal portion of the rotary needle;
inserting the surgical instrument through the penetration in the tissue.

16. The method of claim 15, comprising
inserting an inflatable member into the penetration inflating the inflatable member;
placing a distal end of the endoscope at a proximal end of the inflatable member;
forcing the inflatable member and the distal end of the endoscope through the penetration;
deflating the inflatable member; and
removing the surgical instrument from the working channel of the endoscope.

17. The method of claim 15, wherein the surgical instrument comprises a handle assembly, wherein the rotary needle is advanced distally from the cannula by rotating a portion of the handle assembly.

18. The method of claim 17, wherein the handle assembly comprises a wheel, wherein rotation of the wheel rotates the rotary needle.

19. The method of claim 15, wherein a circular cutting edge is located on the periphery of the distal portion of the rotary needle.

20. The method of claim 15, comprising placing a distal end of the cannula against the portion of tissue to be penetrated.
